(19) United States
(12) Plant Patent Application Publication
(10) Pub. No.: US 2004/0034896 P1
(43) Pub. Date: Feb. 19, 2004

(54) Chrysanthemum plant named raspberry sorbet

(76) Inventor: Nathan Q. Smith, Nipomo, CA (US)

Correspondence Address: BARBARA S. KITCHELL, SUITE 400, 222 LAKEVIEW AVENUE, WEST PALM BEACH, FL, 33401, US

(21) Appl. No.: 10/218,263

(22) Filed: Aug. 13, 2002

(51) Int. Cl. A01H 5/00

(52) U.S. Cl. PLT/298

(57) ABSTRACT

The Raspberry Sorbet is a new and distinct cultivar of chrysanthemum botanically known as Dendranthema grandiflora, and referred to by the cultivar name of Raspberry Sorbet.

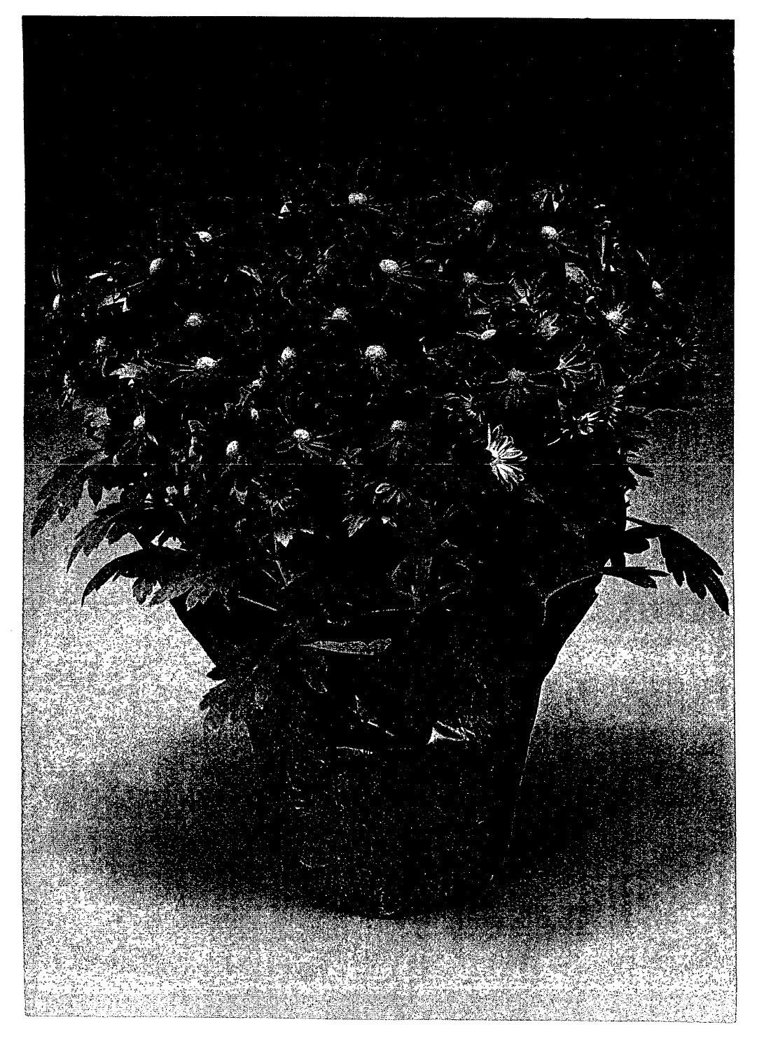

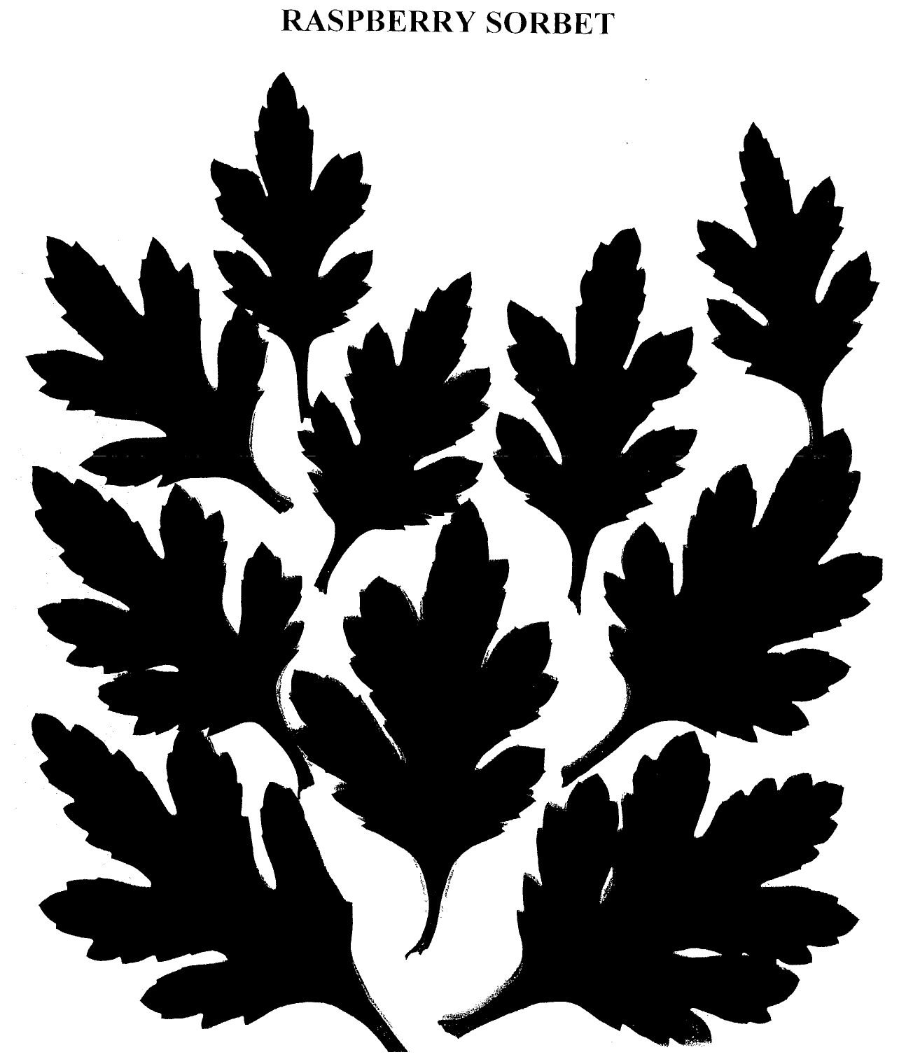

### BACKGROUND OF THE NEW PLANT

The female parent of Raspberry Sorbet was the commercial variety Raspberry Ice, (U.S. Plant Pat. No. 10390) and the male parent was Tijauna, U.S. Plant Pat. No. 9083. The cross was made in the Spring of 1996.

Raspberry Sorbet was discovered and selected as a unique flowering plant among the progeny of the stated cross by Nathan Q. Smith in March 1998 in a controlled environment at Clearwater Nursery, in Nipomo, Calif.

### SUMMARY OF THE INVENTION

Horticultural examination of controlled flowerings of successive plantings has shown that the unique combination of characteristics as herein disclosed for Raspberry Sorbet are firmly fixed and are retained through successive generations of asexual reproduction.

Raspberry Sorbet has not been observed under all possible environmental conditions. The phenotype may vary significantly with variations in the environment such as temperature, light intensity and day length.

The following observations, measurements and comparisons describe plants grown in a controlled environment in Nipomo, Calif. Unrooted cuttings were directly stuck into 6.5″ pots and grown under the natural temperature and day length prevailing in March through July of 2001.

Raspberry Sorbet differs from parents Tijauna and Raspberry Ice, in the following ways:

1. It can be produced year round vs. March through November for Tijauna.

2. It produces a fuller more densely vegetative body than either parent.

3. It spreads more laterally than Tijauna's upright habit.

4. It produces more flowers and branches than either parent.

5. Its ray color is dark purple vs. medium purple rays for Raspberry Ice and Tijauna with white rays that edges graduate out to medium purple.

6. It mature flower reains its color better than either parent variety.

The following traits have been repeatedly observed and are determined to be basic characteristics Raspberry Sorbet, which in combination distinguish this chrysanthemum as a new and distinct cultivar.

1. Flat capitulum form.

2. Daisy capitulum type.

3. Deep purple daisy formed outer ray florets.

4. Yellow disk with green eye.

5. Diameter across the face of the capitulum of 7.0 cm.

6. Exceptional upright to spreading habit with an average of 6.0 breaks after a single pinch.

7. Flowering response is approximately 60 days from the start of short days.

8. Relatively easy to control plant height from bench such that the finished height will range from 16 inches to 18 inches depending on pot size and market requirements.

### DESCRIPTION OF THE DRAWINGS

The accompanying drawings show a typical inflorescence and leaf characteristics of Raspberry Sorbet, with the colors being as nearly true as possible with illustrations of this type.

Sheet 1 is a color photograph Raspberry Sorbet grown as a pinched spray pot mum with 5 cuttings in a 6.5″ pot.

Sheet 2 is a black and white photograph showing the under sides of mature leaves of Raspberry Sorbet.

Of the commercial cultivars known to the inventor, the most similar in form, color, and habit to Raspberry Sorbet is the cultivar Raspberry Ice. Reference is made to “Chart A” which compares certain characteristics of Raspberry Sorbet to the same characteristics of the aforementioned cultivar.

CHART A

| | RASPBERRY | RASPBERRY |
|---|---|---|
| CHARACTERISTIC | SORBET | ICE |
| CAPITULUM FORM | FLAT | FLAT |
| CAPITULUM TYPE | DAISY | DAISY |
| DIAMETER ACROSS | 7.0 CM | 8.1 CM |
| FACE OF THE | | |
| CAPITULUM | | |
| DIAMETER OF DISC | 1.2 CM | 1.36 CM |
| BRANCHING | UPRIGHT AND | UPRIGHT AND |
| PATTERN | SPREADING | SPREADING |
| BREAKS/PINCH | 6 | 5.8 |
| HEIGHT IN A | 16-18″ | 16-18″ |
| 6.5″ POT | | |
| DISC FLORET | EDGE BEGIN | EDGE BEGIN |
| | W/RHS 9A | W/ RHS 9A |
| | GRADUATING TO | GRADUATING TO |
| | YELLOW GREEN | YELLOW GREEN |
| | RHS 144A | RHS 144A |
| # DISC FLORETS | 150 | 151 |
| LENGTH OF DISC | .40 CM | .54 CM |
| FLORETS | | |
| RAY FLORET COLOR | FRONT: | FRONT: |
| | RED-PURPLE | GREYED-PURPLE |
| | RHS 59A | RHS 187D |
| | BACK: | BACK: |
| | GREYED-PURPLE | GREYED-PURPLE |
| | RHS 186B | RHS 185D |
| # RAY FLORETS | 26 | 29 |
| SHAPE OF RAY | ELLIPTIC | ELLIPTIC |
| FLORETS | | |
| LENGTH OF RAY | 3.5 CM | 4.4 CM |
| FLORETS | | |
| WIDTH OF | 1.25 CM | 1.2 CM |
| RAY FLORETS | | |
| NUMBER OF | 6.8 | 6.8 |
| FLOWERS/BREAK | | |
| LEAF COLOR | FRONT: RHS 147 A | FRONT: RHS 147 A |
| | BACK: RHS 137 C | BACK: RHS 147 B |
| MATURE LEAF | 6.5 CM | 4.7 CM |
| WIDTH | | |
| MATURE LEAF | 10.6 CM | 9.5 CM |
| LENGTH | | |
| RESPONSE/ | 8.5 WK | 8.5 WK |
| TREATMENT | | |

Similar traits of the two cultivars include, the flat, daisy capitulum. The diameter across the face of the capitulum of Raspberry Sorbet is smaller than the capitulum face diameter of Raspberry Ice. Both cultivars have similar upright, spreading branching patterns though Raspberry Sorbet is much fuller. Raspberry Ice has fewer breaks per pinch than Raspberry Sorbet. The finished plant height is similar for each cultivar. Raspberry Ice tends to have slightly more disc florets than Raspberry Sorbet. The length of the disc florets is shorter for Raspberry Sorbet vs. Raspberry Ice. Using The ROYAL HORTICULTURAL SOCIETY COLOUR CHART as a standard reference, the front ray floret color for Raspberry Sorbet is Red-Purple 59A while the color of the front ray florets of Raspberry Ice is Greyed-Purple 187D. Raspberry Ice has more ray florets than Raspberry Sorbet. The ray florets of both Raspberry Sorbet and Raspberry ice are elliptic. Raspberry Sorbet has ray florets that are shorter in length than Raspberry Ice. The width of the ray florets of Raspberry Sorbet is greater to those of Raspberry Ice. The color of the leaves of Raspberry Ice is equal to the color of the leaves of Raspberry Sorbet. Raspberry Sorbet has longer and wider mature leaves than Raspberry Ice. Finally, Raspberry Sorbet and Raspberry Ice both have has a response time of 8.5 weeks from the start of short days.

### DESCRIPTION OF THE NEW PLANT

### CLASSIFICATION

Botanical: Dendranthema grandiflora.

CV. name Raspberry Sorbet.

Commercial: Flat daisy spoon to be grown primarily as a SPRAY pot mum.

I. Inflorescence:

A. Capitulum

Form.—Flat.

Type.—Daisy.

Diameter across face.—average of 7.0 cm.

B. Corolla of Ray Florets

Color (general tonality from a distance of three meters).—Red-Purple.

Ray floret color.—Front Fully Expanded Ray Floret: RHS 59A Back Fully Expanded Ray Floret: RHS 186B.

C. Corolla of Disc Florets

Disc floret color.—Edge begins with Yellow RHS 9A to and graduates to center Yellow-Green of RHS 144A.

D. Reproductive Organs

Androecium.—Present in disc florets only, pollen absent.

Gynoecium.—Present both in disc and ray florets.

II. Plant:

A. General Appearance

Height.—When grown in a 6.5″ pot with 1-4 applications of B-9 the total plant height, from the bottom of the pot may be maintained at 16 to 18 inches.

Branching pattern.—Upright and spreading with an average of 6.0 breaks when grown with one pinch.

B. Foliage

Color (upper surface).—RHS 147A.

Color (lower surface).—RHS 137C.

Shape.—See Photograph.

What is claimed is:

1. A new and distinct chrysanthemum cultivar substantially as herein described and illustrated.
